(19) United States
(12) Patent Application Publication
(10) Pub. No.: US 2010/0035773 A1
(43) Pub. Date: Feb. 11, 2010

(54) LUBRICATING OIL COMPOSITION FOR HYDRODYNAMIC BEARING DEVICE AND HYDRODYNAMIC BEARING DEVICE USING SAME

(76) Inventors: Katsushi HIRATA, Ehime (JP); Takanori Shiraishi, Ehime (JP)

Correspondence Address: WENDEROTH, LIND & PONACK L.L.P., 1030 15th Street, N.W., Suite 400 East, Washington, DC, 20005-1503, US

(21) Appl. No.: 12/536,813

(22) Filed: Aug. 6, 2009

(30) Foreign Application Priority Data
Aug. 7, 2008 (JP) 2008-204561

(51) Int. Cl. F16C 32/06; C10M 135/10; C07C 309/00

(52) U.S. Cl. 508/100; 508/390; 562/30

(57) ABSTRACT

The aim of the present invention is to provide a lubricating oil composition for a hydrodynamic bearing device wherein oil film disruption is suppressed. It is solved by a lubricating oil composition for a hydrodynamic bearing device which comprises a base oil and an oil film disruption inhibitor such as metal sulfonate.

### CROSS REFERENCE TO RELATED APPLICATION

The disclosure of Japanese Patent Application No. 2008-204561 filed on Aug. 7, 2008 including the specification, drawings and abstract is incorporated herein by reference in its entirety.

### TECHNICAL FIELD

The present invention relates to a lubricating oil composition for a hydrodynamic bearing device and a hydrodynamic bearing device using same.

### BACKGROUND ART

In recording/reproducing devices that rotate a disk in a hard disk drive or the like, not only is higher-speed rotation necessary, but increased precision in rotation, miniaturization and lower power consumption are required. For this reason, in spindle motors used therein, bearing members in the hydrodynamic bearings can be replaced with parts that are designed for further increased rotation performance, miniaturization and lower cost.

In particular, in the case of the spindle motors used in hard disk drives, furthermore, in order to maintain reliability of the hard disk drive even under harsh conditions such as in high-temperature, high-humidity environments, the capability for stabilized starting currents and stabilized short starting times are is required.

On the other hand, in the spindle motor hydrodynamic bearing device, in addition to the hydrodynamic pressure groove configuration and precision, the properties of the lubrication oil composition have an enormous effect on performance.

Furthermore, the use of barium sulfonates and the like as rust inhibitors in lubricating oil compositions is known. (For example, see Patent Document 1.)

### Prior Art Literature

### Patent Literature

### Patent Document 1

Japanese Published Unexamined Patent Application No. 2004-018531

### SUMMARY OF THE INVENTION

### Problem to be Solved by the Invention

When a hard disk drive spindle motor that uses a hydrodynamic bearing device is left in a high-temperature, high-humidity environment, water content dissolved in the lubricating oil composition is adsorbed onto the metal surface of the bearing, which produces disruption of the oil film of the lubricating oil composition when starting rotation is begun, and consequently the starting current becomes higher, the starting time becomes longer and the like, and performance becomes destabilized.

For this reason, the aim of the present invention is to provide a lubricating oil composition for a hydrodynamic bearing device wherein disruption of the oil film on the bearing metal is suppressed.

### Means to Solve the Problem

Taking account of the aforementioned problem, the present inventors discovered from the results of diligently conducted research that when the general purpose rust inhibitor barium sulfonate was added to a lubricating oil composition, disruption of the oil film of said lubricating oil composition was suppressed, even when left in a high-temperature, high-humidity environment, and the performance of a hard disk drive spindle motor that uses a hydrodynamic bearing device could be stabilized, and with the results of further research were thereupon able to complete the present invention.

Specifically, the present invention relates to items 1 through 9 below.

### Item 1

A lubricating oil composition for a hydrodynamic bearing device which comprises a base oil and metal sulfonate as an oil film disruption inhibitor.

### Item 2

The lubricating oil composition for a hydrodynamic bearing device according to the Item 1, wherein said metal sulfonate is barium sulfonate.

### Item 3

The lubricating oil composition for a hydrodynamic bearing device according to the Item 1, wherein said base oil is an ester oil, an ether oil or hydrocarbon oil or a mixture thereof.

### Item 4

The lubricating oil composition for a hydrodynamic bearing device according to the Item 2, wherein said barium sulfonate is contained in amount 0.2-5 wt % based on the entire composition.

### Item 5

A hydrodynamic bearing device which comprises
a sleeve that possesses a bearing bore, and
a shaft structure being positioned in said bearing bore in a rotatable state relative to said sleeve, and
the lubricating oil composition for a hydrodynamic bearing device according to any one of the Items 1 to 4 which is maintained in the gap formed between said sleeve and said shaft structure.

### Item 6

An oil film disruption inhibitor for a hydrodynamic bearing device lubricating oil composition which comprises a metal sulfonate.

### Item 7

The oil film disruption inhibitor for the hydrodynamic bearing device lubricating oil composition according to the Item 6, wherein said metal sulfonate is barium sulfonate.

### Item 8

A method for preventing disruption of the oil film of a lubricating oil composition for a hydrodynamic bearing device which comprises:
adding metal sulfonate to said lubricating oil composition for a hydrodynamic bearing device.

### Item 9

The method according to the Item 8, wherein said metal sulfonate is barium sulfonate.

### Effect of the Invention

A lubricating oil composition for a hydrodynamic bearing device of the present invention suppresses disruption of the oil film on bearing metal, and a hard disk drive spindle motor that uses a hydrodynamic bearing device that employs said composition will exhibit stabilized performance even in a high-temperature, high-humidity environment.

### BRIEF EXPLANATION OF DIAGRAMS

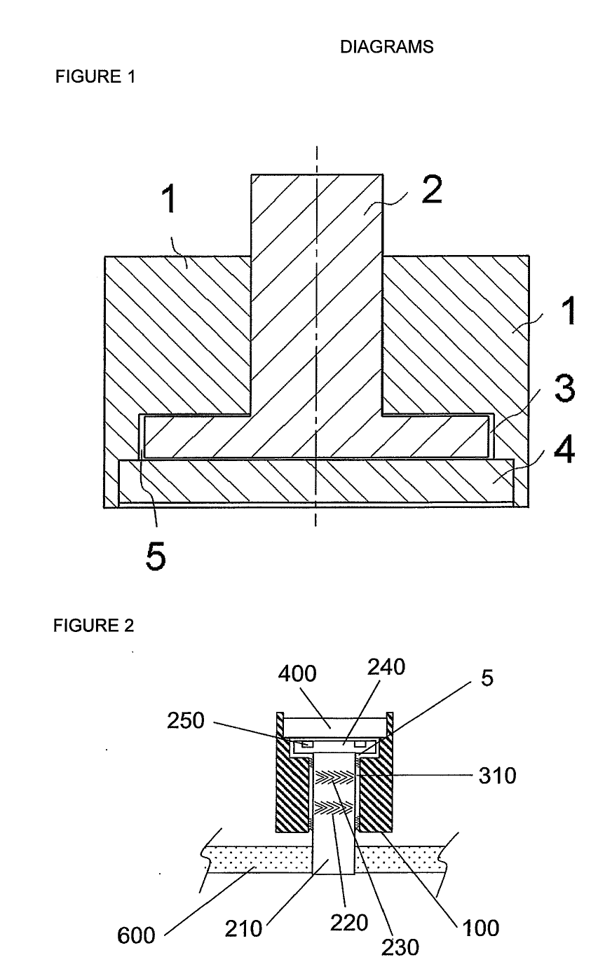

FIG. 1 is a cross-sectional diagram that shows the constitution of the main components of one embodiment of a hydrodynamic bearing device of the present invention.

FIG. 2 is a cross-sectional diagram of the main components of a fixed shaft-type hydrodynamic bearing device of the present invention.

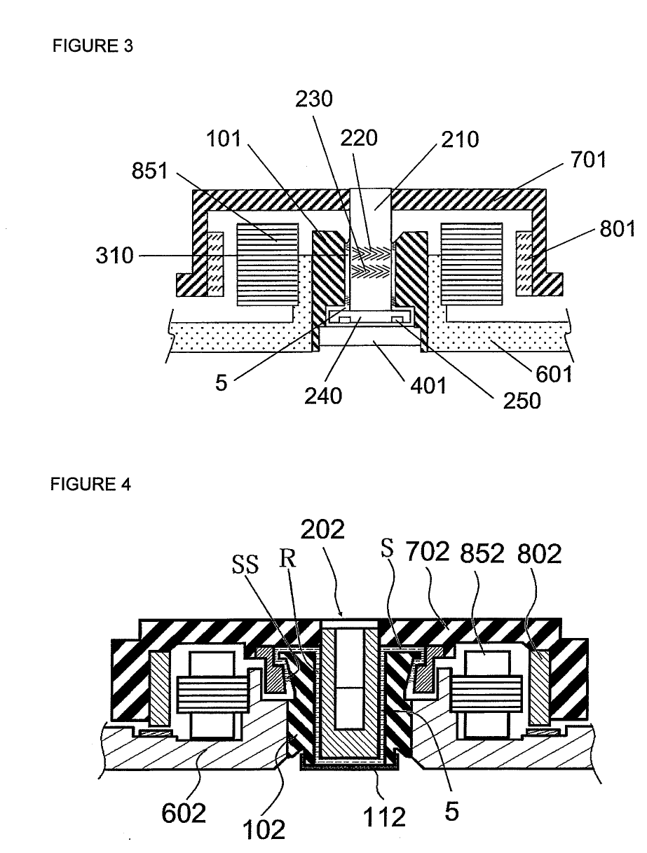

FIG. 3 is a cross-sectional diagram of the main components of a magnetic disk device equipped with a spindle motor that possesses a rotating shaft-type hydrodynamic bearing device of the present invention.

FIG. 4 is a cross-sectional diagram of the main components of a magnetic disk device equipped with a spindle motor that possesses a hydrodynamic bearing device of the present invention.

### MODES FOR IMPLEMENTING THE INVENTION

The present invention is explained below. For the base oil used in the present invention, the base oils commonly used for lubricating oil composition for hydrodynamic bearing devices can be used. Examples of such base oils that can be named include ester oils, ether oils, and hydrocarbon oils as well as mixtures thereof.

Examples of ester oils that can be named include aromatic esters such as trioctyl trimellitate, tridecyl trimellitate, tetraoctyl pyromellitate and the like; monoesters such as esters of monovalent alcohols such as hexanol, 2-ethylhexanol, heptanol, octanol, nonanol, decanol, undecanol, dodecanol, tridecanol, tetradecanol, pentadecanol or hexadecanol or the like with aliphatic monocarboxylic acids such as hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid or octadecanoic acid or the like; diesters (dibasic acid esters) such as dioctyl sebacate (DOS), dioctyl azelate (DOZ), dioctyl adipate (DOA), diisononyl adipate, diisodecyl adipate and the like; as well as polyol esters that are esters of C-5 to C-12 aliphatic acids with alcohols such as neopentyl glycol, polyglycol, 3-methyl-1,5-pentanediol, 2,4-dimethyl-1,5-pentanediol, 2,4-diethyl-1,5-pentanediol, trimethylolethane, trimethylolpropane, pentaerythritol and the like.

Examples of ether oils that can be named include polyglycols such as poly(ethylene glycol), poly(propylene glycol), poly(ethylene glycol)monoether, poly(propylene glycol) monoether and the like, or phenyl ethers such as monoalkyl triphenyl ethers, alkyl diphenyl ethers, dialkyl diphenyl ethers, pentaphenyl ethers, tetraphenyl ethers, monoalkyl tetraphenyl ethers, dialkyl tetraphenyl ethers and the like.

Examples of hydrocarbon oils that can be named include normal paraffins, isoparaffins, polyolefins such as polybutene, 1-decene oligomers, co-oligomers of 1-decene and ethylene, as well as hydrogenation products thereof and the like.

Among these, due to their low viscosity, high heat-resistance, and superior fluidity at low temperatures, the ester oils that are diesters or polyol esters are preferred, and polyol esters are more preferred.

Metal sulfonates preferred for use in the present invention are alkaline earth metal salts or alkali metal salts of sulfonated alkylaromatic compounds, with a weight-average molecular weight of 100-1500, preferably 400-1200. More specifically, examples of alkaline earth metal salts or alkali metal salts that can be named include barium sulfonates, calcium sulfonates, sodium sulfonates, potassium sulfonates, lithium sulfonates and the like.

Among these, alkaline earth metal salts such as barium sulfonates, calcium sulfonates and the like are preferred. Alkaline earth metal salts are metal salts that are bonded to divalent metal elements, and since the molecular weights are higher than for metal salts that are bonded to monovalent metal elements, are more preferred from the perspectives of increased heat resistance and effective prevention of oil film disruption.

In addition, either petroleum-based metal sulfonates or synthetic metal sulfonates are satisfactory. Petroleum-based metal sulfonates are obtained by sulfonation of a petroleum fraction raw material, and synthetic metal sulfonates are obtained by sulfonation of synthetic alkylbenzenes.

Furthermore, petroleum-based metal sulfonates and synthetic metal sulfonates can be diluted with refined mineral oil or synthetic oil or the like, but the metal sulfonate content is preferably ≧30 wt % of the total, more preferably ≧50 wt %.

Among these, petroleum-based barium sulfonates or synthetic barium sulfonates are preferred. In particular, due to the narrow molecular weight distribution, synthetic barium sulfonates are more preferred from the perspectives of increased oxidation stability and increased heat resistance.

Metal sulfonates that are utilized commercially as rust inhibitors can be used.

Specific examples of barium sulfonates that can be named include NA-SUL BSN (King); Barinate B-70, Basic Barium Petronate, Neutral Barium Petronate, Surchem 404, Surchem 404D (Chemtura), Sulfol Ba-30N, Moresco-amber SB-50N, Moresco-amber APC, Moresco-amber OPC (Matsumura Oil Research Corp.) and the like.

The weight-average molecular weights for these are preferably 1000-1100. Furthermore, the preferred barium sulfonate content when diluted with refined petroleum, synthetic oil or the like is ≧30 wt % of the total, and more preferably is ≧50 wt %.

For a lubricating oil composition for a hydrodynamic bearing device of the present invention, the abovementioned metal sulfonate is preferably contained in amount of 0.2-5 wt % (more preferably 0.2-2 wt %, further preferably 0.3-1 wt %) based on the total composition. Moreover, in the present specification, without being limited in any particular way to such a description, wt % means % (w/w).

The addition of a metal sulfonate to a lubricating oil composition for a hydrodynamic bearing device provides a grounding effect for static charge that has accumulated on the device due to a conductive property having been conferred (reducing the volume resistivity), and a surface adsorption effect, and for uncured components of the epoxy adhesive that are oil sealing agents in a hydrodynamic bearing device, an effect of suppressing the leaching of extremely small quantities thereof from the lubricating oil composition due to changes in polarity of the lubricating oil in addition to the abovementioned effect of suppressing oil film disruption.

Since a higher metal sulfonate content will not increase the effects of added metal sulfonate, it is not preferred on economic grounds, and not preferred because of increases in viscosity and in the current during starting and stable rotation. This is furthermore unsuitable because of separating or turbidity occurs at low temperatures below room temperature, reduction in the frictional and wear characteristics of the bearing device, and concerns that rotation failure (lock) will occur.

On the other hand, if the metal sulfonate content is lower, an inadequate effect will be obtained from the added metal sulfonate.

The lubricating oil composition for a hydrodynamic bearing device of the present invention can also contain additives conventionally used in lubricating oil compositions for hydrodynamic bearing devices other than metal sulfonates.

Examples of such additives that can be named include antioxidants, rust inhibitors, metal deactivators, metal corrosion inhibitors, oiliness improvers, extreme pressure agents, friction modifiers, anti-wear agents, viscosity index improvers, pour point depressants, antifoaming agents, hydrolysis inhibitors, antistatic agents, conductivity-enhancing agents, detergent dispersants and the like. Among these, regarding metal deactivators, rust inhibitors, antistatic agents, conductivity-enhancing agents, detergent dispersants and the like, since metal sulfonates possess all of such properties, either these are not added or the amounts added can be curtailed.

When such additives are added, their amounts are preferably ≦5 wt % (more preferably ≦4 wt %, further preferably ≦3 wt %), based on the entire composition. Inter alia, antioxidants suppress the oxidative degradation of the lubricating oil composition for a hydrodynamic bearing device and are essential for increasing the heat resistance. Specifically, antioxidants of the phenol type or of the amine type that do not contain sulfur or chlorine in the molecule are the most suitable for inhibiting oxidation. If additives that contain sulfur or chlorine in the molecule undergo decomposition, corrosive gases will be generated, and there is a concern that these would exert a significant effect on the performance of the device. Such antioxidants can be used singly or in combination. Among these, phenol-type antioxidants that possess 2 or more phenol groups are preferred for achieving and maintaining adequate effectiveness even when used in a device in a high temperature environment of 80-100° C. or higher, and for having high heat resistance. Furthermore, it is preferable to select and use liquid type antioxidants so that fluidity is not decreased at low temperatures and starting rotation of the device is easy. The amount of antioxidants added is 0.1-3 wt %, preferably 0.5-3 wt %.

The content of base oil in the lubricating oil composition for a hydrodynamic bearing device of the present invention, based on the weight of total composition but excluding the weight of the content of such metal sulfonates and the abovementioned additives, usually is ≧90 wt %, more preferably is ≧94 wt %, and further preferably is ≧96 wt %.

A lubricating oil composition for a hydrodynamic bearing device of the present invention preferably has the following physicochemical properties: A volume resistivity of ≦1×10¹¹ Ω·cm, preferably ≦1×10¹⁰ Ω·cm, at 20° C. and 5V, measured according to JIS C 2101.

A viscosity index of ≧100, preferably ≧120, and further preferably ≧140, measured according to JIS K 2283.

An amount of evaporation of ≦5 wt %, preferably ≦4 wt %, and more preferably ≦3 wt %, measured according to JIS C 2101.

A pour point of ≦−20° C., preferably ≦−30° C., and more preferably ≦−40° C., measured according to JIS K 2269. Moreover, a low-temperature solidification point of ≦−20° C., preferably ≦−30° C., and more preferably ≦−40° C. It is noted that the low-temperature solidification point in this case is a different temperature from the pour point. The low-temperature solidification point is the temperature at which all or part of the lubricating oil sample in a cup solidifies after being allowed to stand in a thermal bath for 2 days after collection of the lubricating agent in a sample cup, and will be several to several tens of ° C. higher than the pour point.

The kinetic viscosity measured according to JIS K 2283 and the viscosity determined from the density measured according to JIS K 2249 at −20° C. is 70-200 mPa·sec, more preferably 70-150 mPa·sec; at 20° C. is 5-35 mPa·sec, more preferably 10-25 mPa·sec; and, at 80° C. is 2-5 mPa·sec, more preferably 3-4 mPa·sec.

A lubricating oil composition for a hydrodynamic bearing device of the present invention can be manufactured by mixing base oil, metal sulfonate and any optionally added additives using conventional methods for the manufacture of lubricating oils for a hydrodynamic bearing device.

Furthermore, although a lubricating oil composition for a hydrodynamic bearing device of the present invention is suitable for use in a hydrodynamic bearing device, it can also be used for other applications.

In addition, for a lubricating oil composition for a hydrodynamic bearing device of the present invention, it is desirable to carry out a filtration under reduced or pressurized pressure before filling into a hydrodynamic bearing device with a filter that has a pore diameter smaller than the minimum clearance formed between the sleeve and the shaft structure. Consequently, foreign matter is removed, which can suppress the rotation failure caused by foreign matter. Moreover, specifically the filter pore diameter is ≦0.5 μm, preferably ≦0.2 μm, and more preferably ≦0.1 μm.

The oil film disruption inhibitor of the lubricating oil composition for a hydrodynamic bearing device can consist of metal sulfonate or comprise metal sulfonate. Said oil film disruption inhibitor can be contained in an additive used in conventional lubricating oil compositions for hydrodynamic bearing devices as mentioned above, and consequently can impart desired effects other than preventing oil film disruption.

The relative amount of the additive with respect to the metal sulfonate in this case can be determined in the same manner as for the components in the abovementioned conventional lubricating oil composition for a hydrodynamic bearing device.

For example, by adding an oil film disruption inhibitor of the present invention to a commercial lubricating oil composition used in hydrodynamic bearing devices, and mixing, oil film disruption can be prevented.

A lubricating oil composition used in hydrodynamic bearing devices of the present invention can be used in all hydrodynamic bearing devices. Moreover, a hydrodynamic bearing device that uses a lubricating oil composition used in hydrodynamic bearing devices of the present invention is also one mode of the present invention.

For the purpose of explaining an overview of the hydrodynamic bearing device of the present invention, one mode thereof is shown in FIG. 1, but the present invention is not limited thereto.

Said hydrodynamic bearing device possesses sleeve 1 that possesses a bearing bore and shaft structure 2 that is positioned in a relatively rotatable state with respect to aforementioned sleeve 1 within aforementioned bearing bore, and hydrodynamic bearing device lubricating oil composition 5 of the present invention is maintained in gap 3 between aforementioned sleeve 1 aforementioned shaft structure 2.

The best embodiments of the present invention are shown in detail below, and are described together with the diagrams.

### Embodiment 1

Embodiment 1 of the present invention is explained by using FIG. 2. FIG. 2 is a cross-sectional diagram of the main components of a fixed shaft type hydrodynamic bearing device in Embodiment 1.

In FIG. 2, radial dynamic pressure-generating grooves 220, 230 are formed on the outer peripheral surface of shaft 210. One end of shaft 210 is affixed to thrust flange 240 and the other end is press-fitted and affixed to base 600. Shaft 210 and thrust flange 240 constitute a shaft structure. The shaft structure and base 600 constitute a fixed portion.

At the same time, sleeve 100 has a bearing bore that supports the shaft structure. Thrust plate 400 is mounted on one end of sleeve 100. The shaft structure is inserted into the bearing bore of sleeve 100 so that thrust plate 400 and thrust flange 240 face each other. Sleeve 100 and thrust plate 400 constitute a rotator. In addition, thrust dynamic pressure-generating groove 250 is formed at the facing surfaces of thrust flange 240 and thrust plate 400. Hydrodynamic bearing device lubricating oil composition 5 of the present invention is interposed into the gap between the bearing bore and the shaft structure. A motor drive portion is formed by the rotator and the fixed portion.

Accompanying the rotation of the rotator, hydrodynamic bearing device lubricating oil composition 5 is gathered up in dynamic pressure-generating grooves 220, 230, which generate pumping pressure in the radial direction in radial gap 310 between shaft 210 and sleeve 100. In the same manner, due to the rotation, hydrodynamic bearing device lubricating oil composition 5 is gathered up in dynamic pressure-generating groove 250, which generate pumping pressure in the thrust direction between thrust flange 240 and thrust plate 400. In this way, the rotator is floated with respect to the fixed portion and is rotatably supported without contact.

Furthermore, as mentioned in the explanation above, radial dynamic pressure-generating grooves are formed on the outer peripheral surface of shaft 210, but they can also be formed on the bearing bore surface of sleeve 100 (inner peripheral surface), as well as on both the outer peripheral surface of shaft 210 and the bearing bore surface of sleeve 100. In other words, at least one of the shaft and the sleeve can have radial dynamic pressure-generating mechanical features. Additionally, radial dynamic pressure-generating mechanical features can be present between the lateral surface of thrust flange 240 and sleeve 100. Examples of dynamic pressure-generating mechanical features that can be named include various types of shapes such as grooves, projections, bumps, inclined planes and the like. Moreover, radial dynamic pressure-generating grooves can adopt various types of shapes such as a herringbone shape, a spiral shape and the like (in the diagram, radial dynamic pressure-generating grooves with a herringbone shape are shown).

In addition, thrust dynamic pressure-generating grooves can be formed either only on the face of thrust plate 400 opposite to thrust flange 240, or only the face of thrust flange 240 opposite to thrust plate 400, or only the reverse side of the face of thrust flange 240 opposite to thrust plate 400, as well as on 2 or more of the aforementioned 3 locations.

Furthermore, for any dynamic pressure-generating mechanical features similar to those mentioned above in addition to thrust dynamic pressure-generating grooves, any type of mechanical feature will be satisfactory.

In the present embodiment, one end of the hydrodynamic bearing is fixed, but there is no limitation [to this configuration], and the same effect can be obtained with both ends being fixed or with both ends of the bearing bore of the sleeve being open.

### Embodiment 2

Embodiment 2 of the present invention is explained by using FIG. 3. FIG. 3 is a cross-sectional diagram of the main components of a magnetic disk device equipped with a spindle motor that possesses a rotating shaft-type hydrodynamic bearing device of Embodiment 2. The hydrodynamic bearing device in the present embodiment differs from the hydrodynamic bearing device of Embodiment 1 in FIG. 2 from the perspective of adopting a rotating shaft system that replaces the fixed shaft. Other than this point, the constitution is the same as in Embodiment 1. Furthermore, components that have the same symbols are omitted in the detailed explanation.

In FIG. 3, radial dynamic pressure-generating grooves 220, 230 are formed in the outer peripheral surface of shaft 210, one end of which is affixed to thrust flange 240 and the other end of which is pressure-fitted into hub 701 for mounting a magnetic disk. Shaft 210 and thrust flange 240 form the shaft structure. Moreover, rotor magnet 801 is affixed to the inner peripheral surface of hub 701. The shaft structure (shaft 210 and thrust flange 240), hub 701 and rotor magnet 801 constitute the rotator. Furthermore, in the present invention, the shaft structure can be constituted from shaft 210 alone, or the shaft structure can be constituted from shaft 210 and thrust flange 240 as desired.

At the same time, sleeve 101 that is pressure-fitted into base 601 has a bearing bore that supports the shaft structure. Thrust plate 401 is mounted on one end of sleeve 101. The shaft structure is inserted into the bearing bore of sleeve 101 so that thrust plate 401 and thrust flange 240 face each other. Stator coil 851 is mounted on a wall formed by base 601. Base 601, sleeve 101, thrust plate 401 and stator coil 851 form the fixed portion. Thrust dynamic pressure-generating groove 250 is formed at the facing surfaces of thrust flange 240 and thrust plate 401. The bearing device is constituted when hydrodynamic bearing device lubricating oil composition 5 is filled into the gap between the bearing bore and the shaft structure. The rotator and the fixed portion constitute the motor drive component.

The rotational driving action of the rotator due to this motor drive component will be explained.

First, stator coil 851 is energized to produce a rotating magnetic field, and rotor magnet 801 that is mounted to face stator coil 851 experiences rotational force and hub 701, shaft 210 and thrust flange 240 begin to rotate together. Due to this rotation, herringbone-shaped dynamic pressure-generating grooves 220, 230 and 250 gather up hydrodynamic bearing device lubricating oil composition 5, and pumping pressure is generated in the radial direction together with in the thrust direction (between shaft 210 and sleeve 101, and between thrust flange 240 and thrust plate 401). As a result, the rotator is floated with respect to the fixed portion and is rotatably supported without contact, and recording and reproduction of data on the magnetic disk is possible.

Furthermore, without being limiting in any way, the material of magnetic disk 11 mounted on hub 701 can be glass or aluminum, and in the case of small-scale machine types, without being limiting in any way, usually ≧1 disk (usually 1-2 disks) is attached. Among these, magnetic disk devices and spindle motors equipped with small-scale magnetic disks ≦2.5 inches in size are effective for the present invention.

### Embodiment 3

FIG. 4 is a cross-sectional drawing of the main components of a magnetic disk device equipped with a spindle motor that has a rotating shaft-type hydrodynamic bearing device of Embodiment 3.

In this magnetic disk device, sleeve 102 that possesses a bearing bore that supports shaft structure 202 is pressure-fitted into the center of base 602, and stator coil 852 is mounted on a wall formed on base 602. Shaft structure 202 is inserted from one lateral end into the bearing bore of sleeve 102 and the other end is blocked by cap 112. Radial dynamic pressure-generating grooves (not shown in the figure) are formed on the outer peripheral surface of shaft structure 202, and one of its ends is pressure-fitted into hub 702 while the other end faces cap 112. The outer peripheral surface (dynamic pressure surface) of shaft structure 202 passes through gap R in the radial direction with respect to the inner peripheral surface (dynamic pressure surface) of sleeve 102 and faces thereto, and this gap R is filled with hydrodynamic bearing device lubricating oil composition 5. Rotor magnet 802 is affixed to the inner peripheral surface of hub 702.

Additionally, the top end surface (dynamic pressure surface) of sleeve 102 and the bottom end surface (dynamic pressure surface) in the interior side of hub 702 are positioned to face each other passing through gap S in the axial direction, and thrust dynamic pressure-generating grooves (not shown in the figure) are formed on at least one side of these surfaces. For filling also gap S, hydrodynamic bearing device lubricating oil composition 5 is filled from abovementioned gap R through to gap S in a substantially connected and uninterrupted fashion.

When shaft structure 202 and hub 702 are rotating, dynamic pressure is generated in hydrodynamic bearing device lubricating oil composition 5 due to the action of the abovementioned thrust dynamic pressure-generating grooves. Due to this dynamic pressure, shaft structure 202 and hub 702 are floated in the thrust direction and are rotatably supported without contact.

The outer peripheral side of sleeve 102 forms seal portion SS. The gap of seal portion SS is connected to gap S on the outside along the radial outward direction of sleeve 102, which expands downward. Consequently, seal portion SS prevents the outflow of hydrodynamic bearing device lubricating oil composition 5.

Furthermore, in the abovementioned embodiment, motor rotational speeds of 3,600 rpm, 4,200 rpm, 5,400 rpm, 7,200 rpm, 10,000 rpm, 15,000 rpm or the like are used.

For the material of the shaft, stainless steel is the most suitable. Stainless steel has high hardness compared to other metals, and is effective because wear products are suppressed. More preferable is martensite stainless steel.

For the sleeve, the use of a material such as copper alloy, iron alloy, stainless steel, ceramic, or resin is preferred. In addition, copper alloy, iron alloy or stainless steel are further preferred for greater wear resistance and higher workability, as well having a lower cost. Moreover, sintered materials are also satisfactory from the cost perspective, and the same effect can be obtained when a dynamic pressure-generating liquid is impregnated into a sintered material. For the materials that constitute the bearing such as the shaft material, sleeve material, flange material, thrust plate material and the like, a plating process, physical vapor deposition method, chemical vapor deposition method, diffusion coating method or the like with a material different from the parent material [can be used] to carry out surface modification or surface treatment of a portion of the surface or the entire surface.

### WORKING EXAMPLES

The present invention is explained in further detail below using working examples and comparative examples, although the present invention is not limited thereto.

In the lubrication oil compositions used for hydrodynamic bearings used in the working examples and comparative examples, the ester formed from 3-methyl-1,5-pentandiol and n-octanoic acid is used as the base oil.

Moreover, Moresco-amber SB-50N (Matsumura Oil Research Corp., weight-average molecular weight of 1050, barium sulfonate content of 50 wt %) is used as the synthetic barium sulfonate, and Neutral Barium Petronate (Chemtura, weight-average molecular weight of 1000, barium sulfonate content of 43 wt %) is used as the petroleum-based barium sulfonate.

Additionally, both in the working examples and comparative examples, 0.5 wt % of dioctyl-diphenylamine is blended in as an antioxidant. Moreover, the blending quantities of additives shown in the present invention, in other words the wt % s, are the percentages based on the lubricating oil composition that includes the base oil and the additives (total weight).

### Working Example 1

A lubricating oil for a hydrodynamic bearing device was prepared by adding to the base oil 0.3 wt % of the synthetic barium sulfonate as an oil film disruption inhibitor.

### Working Example 2

A lubricating oil for a hydrodynamic bearing device was prepared by adding to the base oil 1 wt % of the synthetic barium sulfonate as an oil film disruption inhibitor.

### Working Example 3

A lubricating oil for a hydrodynamic bearing device was prepared by adding to the base oil 2 wt % of the synthetic barium sulfonate as an oil film disruption inhibitor.

### Working Example 4

A lubricating oil for a hydrodynamic bearing device was prepared by adding to the base oil 3 wt % of the synthetic barium sulfonate as an oil film disruption inhibitor.

### Working Example 5

A lubricating oil for a hydrodynamic bearing device was prepared by adding to the base oil 2 wt % of the petroleum-based barium sulfonate as an oil film disruption inhibitor.

### Comparative Example 1

For the comparative example, the base oil was used as a lubricating oil for a hydrodynamic bearing device.

### Comparative Example 2

A lubricating oil for a hydrodynamic bearing device was prepared by adding to the base oil 0.1 wt % of the synthetic barium sulfonate as an oil film disruption inhibitor.

### Comparative Example 3

A lubricating oil for a hydrodynamic bearing device was prepared by adding to the base oil 1 wt % of sorbitan trioleate.

Evaluations were performed on the motor starting currents for a bearing device that uses the hydrodynamic bearing device lubricating oils from these Working Examples 1 through 5 and Comparative Examples 1 through 3, and on the volume resistivity of the lubricating oils. The details and results of this evaluation are shown in Table 1.

Moreover, the hydrodynamic bearing device used in the evaluation experiments was a 1.8-inch rotating shaft type in the condition of having 1 magnetic disk attached in a clamp, and the measurement was carried out at 3,600 rpm.

### 1) Motor Starting Current

For the initial starting rotation current, after being left for 1 week under conditions of 85° C. and 90% RH, the starting rotation current was measured at 25° C., and the amount of change (amount of increase) was calculated.

### 2) Volume Resistivity

Measured at 20° C. and 5 V, according to JIS C 2101.

TABLE 1

| | | Motor starting current: | |
|---|---|---|---|
| | | amount of change | |
| | | from initial value | Volume resistivity |
| | | (mA) | (Ω · cm) |
| Working examples | 1 | 5 | 2.0 × 10¹⁰ |
| | 2 | 3 | 3.4 × 10⁹ |
| | 3 | 4 | 2.0 × 10⁹ |
| | 4 | 2 | 1.6 × 10⁹ |
| | 5 | 3 | 3.6 × 10⁹ |
| Comparative examples | 1 | 45 | 6.0 × 10¹¹ |
| | 2 | 37 | 1.0 × 10¹¹ |
| | 3 | 37 | 1.1 × 10¹¹ |

For the motor starting currents using the lubricating oils for hydrodynamic bearings of Working Examples 1 through 5, there was practically no increase from the initial starting currents, and the starting characteristics were stabilized.

At the same time, using the lubricating oils for hydrodynamic bearings of Comparative Examples 1 through 3, the motor starting currents had clearly increased from the initial starting currents, and the starting characteristics had been diminished. Consequently, it was observed that oil film disruption phenomena had occurred.

It is clear from the above that with the hydrodynamic bearing device lubricating oils and the hydrodynamic bearing devices that use same of the present invention, oil film disruption on metal bearings is suppressed, even after being left in a high-temperature, high-humidity environment, and they can exhibit stabilized starting rotation characteristics.

### INDUSTRIAL APPLICABILITY

The hydrodynamic bearing device lubricating oil compositions and the hydrodynamic bearing devices using the same that relate to the present invention can be applied in motors for hard disk drive (magnetic disk devices) information devices, optical disk devices, scanner devices, laser beam printers, video recorders and the like. In particular, they are effective in small-scale hard disk drives that are 2.5 inches or less in size.

1. A lubricating oil composition for a hydrodynamic bearing device which comprises a base oil and metal sulfonate as an oil film disruption inhibitor.

2. The lubricating oil composition for a hydrodynamic bearing device according to claim 1, wherein said metal sulfonate is barium sulfonate.

3. The lubricating oil composition for a hydrodynamic bearing device according to claim 1, wherein said base oil is an ester oil, an ether oil, a hydrocarbon oil, or a mixture thereof.

4. The lubricating oil composition for a hydrodynamic bearing device according to claim 2, wherein said barium sulfonate is contained in amount of 0.2-5 wt % based on the entire composition.

5. A hydrodynamic bearing device which comprises
a sleeve that possesses a bearing bore,
a shaft structure being positioned in said bearing bore in a rotatable state relative to said sleeve, and
the lubricating oil composition for a hydrodynamic bearing device according to claim 1 which is maintained in the gap formed between said sleeve and said shaft structure.

6. An oil film disruption inhibitor for a hydrodynamic bearing device lubricating oil composition which comprises metal sulfonate.

7. The oil film disruption inhibitor for the hydrodynamic bearing device lubricating oil composition according to claim 6, wherein said metal sulfonate is barium sulfonate.

8. A method for preventing disruption of an oil film of a lubricating oil composition for a hydrodynamic bearing device which comprises:
adding metal sulfonate to said lubricating oil composition.

9. The method according to claim 8, wherein said metal sulfonate is barium sulfonate.
